(19) United States
(12) Patent Application Publication
(10) Pub. No.: US 2009/0211029 A1
(43) Pub. Date: Aug. 27, 2009

(54) DEVICE AND METHOD OF LIMB SUPPORT FOR A PATIENT

(76) Inventor: Bongfen Siona Jaff Forba, Hanover Park, IL (US)

Correspondence Address: Mathew R.P. Perrone, Jr.;Brie A. Crawford, 210 South Main Street, Algonquin, IL, 60102, US

(21) Appl. No.: 12/036,480

(22) Filed: Feb. 25, 2008

(51) Int. Cl. A47C 20/00; A61F 5/37; A61F 5/00

(52) U.S. Cl. 5/630; 128/870; 602/4

(57) ABSTRACT

A limb support for a patient in a hospital bed, on the operating table, in a wheel chair, with an intravenous line or in another position for health care services is positioned in the desired area, or removed therefrom due to mounting clips secured to each end of a sling band.

This invention relates to a device and method of limb support for a patient in bed and more particularly to a device and method of limb support for a patient, which facilitates caring for the patient.

### BACKGROUND OF THE INVENTION

With a patient in a hospital, service to the patient by various medical professionals is required. This is especially true when a patient needs a bandage to be changed. It also applies for other medical procedures. For example, insertion of intra venous set up into a patient can be a problem. Even patient comfort, such as reading, requires support.

For a bandage on an arm or leg, support of that limb is required during the changing procedure or applying procedure. Usually, either procedure with the bandage requires two people. It is very desirable to reduce the requirements for two people being used to change a bandage. Yet, the lack of mobility for the typical hospital patient almost always makes changing a bandage a two-person job. If a simple device or method can be used to assist a patient, it becomes more possible for one person to effectively change a bandage for a patient.

Limb support is also required for other medical procedures. One common procedure is an intravenous treatment with the insertion or the removal of an appropriate intravenous device. Two people can greatly facilitate that process.

With help from an appropriate device, even this procedure, which requires great skill, may be handled more efficiently.

Keeping the mind of a patient occupied is also an advantage. Use of hands or mind can help. If activities like reading or crafts can be accomplished efficiently, the patient becomes more satisfied.

Such a device must be easily positioned, easily used and easily removed. No such device is really available. To that end, some devices have been made. However, those devices tend to be too complicated to use. The various factors required to make such a device conflict with each other.

To minimize the factors that conflict with making an effective, easy patient support, especially in the field of bandages or dressings, offers many advantages. Not only will critical time be saved, but the patient will be treated more efficiently.

### SUMMARY OF THE INVENTION

Among the many objectives of the present invention is the provision of a limb support for use with a hospital bed.

Another objective of the present invention is the provision of a limb support attachable to opposing rails of a hospital bed.

Yet another objective of the present invention is the provision of a limb support suit for use with intravenous therapy.

Still another objective of the present invention is the provision of a limb support suitable for use with a wheel chair.

Also, an objective of the present invention is the provision of a limb support easily attachable to opposing rails of a hospital bed.

Moreover, an objective of the present invention is the provision of a limb support easily removable from opposing rails of a hospital bed.

A further objective of the present invention is the provision of a limb support to facilitate applying a bandage to a patient.

A still further objective of the present invention is the provision of a limb support to facilitate changing the bandage for a patient.

Yet further objective of the present invention is the provision of a limb support to facilitate use of an operating table.

These and other objectives of the invention (which other objectives become clear by consideration of the specification, claims and drawings as a whole) are met by providing a limb support for a patient undergoing medical care, with the limb support having sling with a fastener at each thereof for use on a patient in a hospital bed, on the operating table, in a wheel chair or in another medical situation, in order to facilitate treatment of the patient.

### BRIEF DESCRIPTION OF DRAWINGS

FIG. 1 depicts a perspective view of a limb support 100 of this invention, as mounted on a hospital bed 110.

FIG. 2 depicts a top plan view of a limb support 100 of this invention, based on FIG. 1.

FIG. 3 depicts a perspective, close-up, end view of limb support 100 of this invention using a clamp fastener 104.

FIG. 4 depicts a perspective, close-up, end view of limb support 100 of this invention using a hook and loop assembly 124 as a fastener.

FIG. 5 depicts a block diagram of the limb support 100 of this invention on a hospital bed 110.

FIG. 6 depicts a top plan view of limb support 100 of this invention, used on arm 128.

FIG. 7 depicts a perspective, close-up, end view of limb support 100 of this invention using a catch mount 130 as a fastener.

FIG. 8 depicts a block diagram of the limb support 100 of this invention on a mounting surface 170.

Throughout the figures of the drawings, where the same part appears in more than one figure of the drawings, the same number is applied thereto.

### DESCRIPTION OF THE PREFERRED EMBODIMENTS

A medical support device or a limb support is used to temporarily suspend an arm or a leg of a patient confined to a hospital bed, a wheel chair, or another hospital apparatus or home medical apparatus, in to facilitate a plurality of treatments for the patient. Whether it is a limb support or medical support device, which terms are interchangeable unless otherwise specified, care of a patient in a hospital bed or in another hospital apparatus is easier.

One treatment involves bandages or dressings; and includes applying, removing or changing a bandage for a patient in the bed, especially on a limb such as an arm or a leg. The limb support can also assist with other patient services. Such a support facilitates use of another hospital apparatus, including but not limited to, intravenous devices. With proper end support, this limb support is usable in the operating theater. Furthermore, it may be used with a wheelchair, a hospital bed or other suitable device as a support for reading material, game material, craft material, and the like.

As structured, this limb support includes a partially flexible material area or sling band with a sufficient width to support the limb and sufficient length to be mounted as desired. Attached to each end of the material is a rail attachment. The rail attachment provides for each end of the limb support to be attached to opposing rails usually present on a hospital bed or other support.

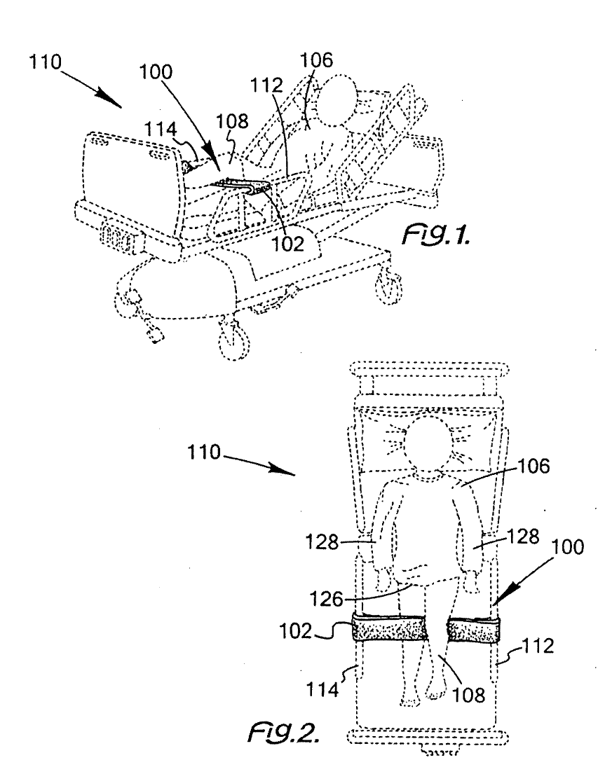

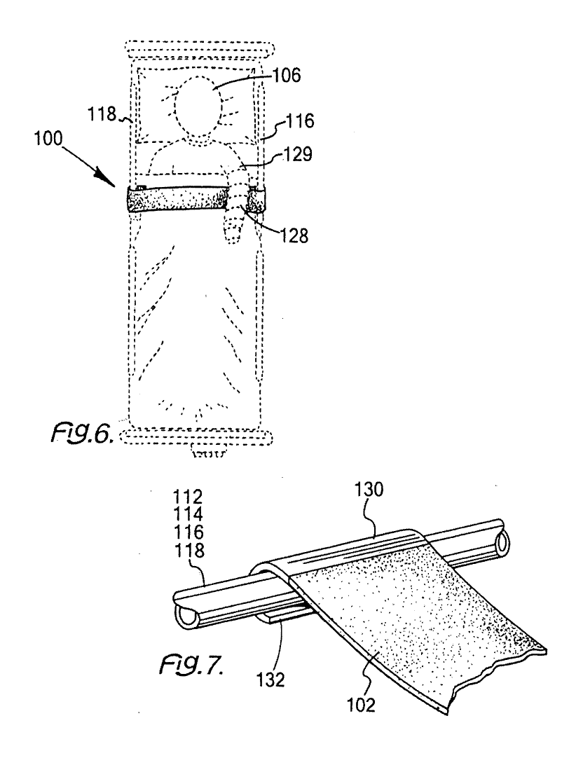

In FIG. 1 and FIG. 2, limb support 100 is secured to a hospital bed 110 in a releasable fashion. The hospital bed 110 usually has a first right rail 112 and a first left rail 114 mounted on the sides thereof by the hips or legs 126 of the patient 106. There may also be a second right rail 116 and a second left rail 118 by the arms 128 or shoulders 129 of the patient 106 (FIG. 6. The limb support 100 of this invention cooperates with the rails 112 and 114 or rails 116 and 118 support an arm 128 during a bandage change or application. In this particular case, a leg 108 is supported as desired.

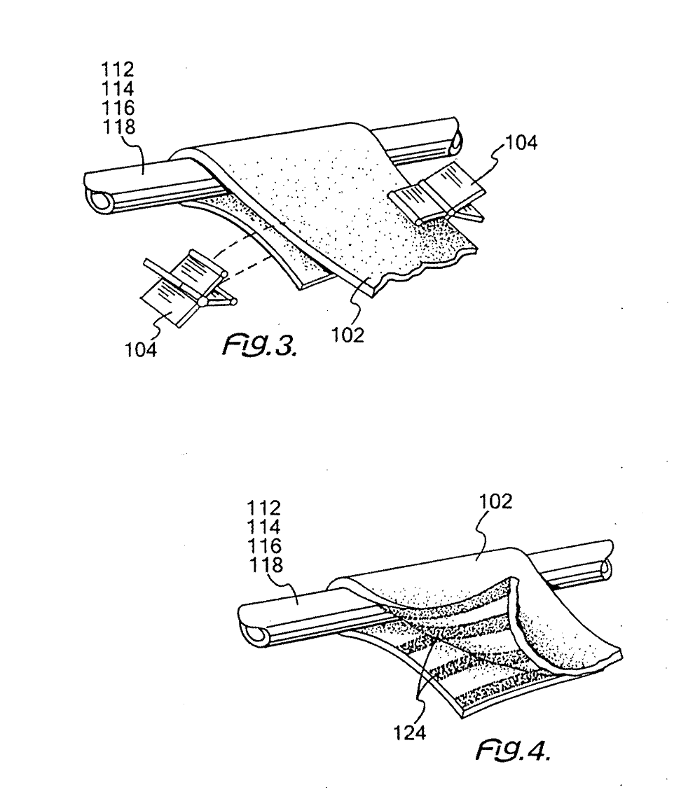

Now considering FIG. 3, limb support 100 has a sling band 102, which may be releasably secured to the rails 112 and 114 or rails 116 and 118, by folding the same thereover and apply a pair of spring clamps 104 on opposing sides of double layer of the sling band 102 formed by wrapping each end of the sling band 102 around the rails 112 and 114 or rails 116 and 118. Spring clamps 104 are easily applied or removed as desired.

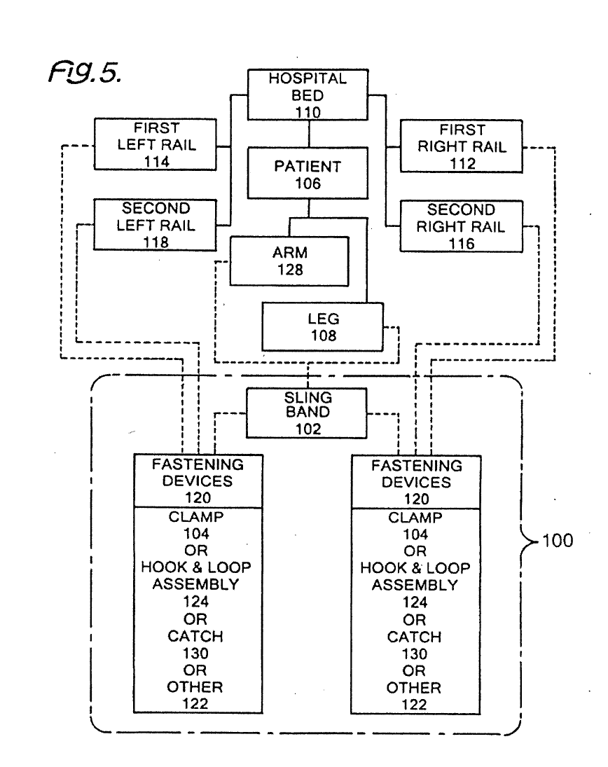

Still adding FIG. 4 to the consideration, limb support 100 as sling band 102 secured to the rails 112 and 114 or rails 116 and 118 by a hook and loop assembly 124. The hook and loop assembly 124 also permits the sling band 102 and limb support 100 to be easily secured or removed as desired.

A standard hook and loop assembly is available under the registered trademark VELCRO, owned by Velcro Industries B.V. LIMITED LIABILITY COMPANY NETHERLANDS Castorweg 22-24 Curacao NETHERLANDS ANTILLES.

Now adding FIG. 5 to the consideration, limb support 100 is depicted as supporting the arm 128 or the leg 108 in a hospital bed 110. Sling band 102 is somewhat rigid and permits a variety of fastening devices 120 to be secured to each end thereof in order to form limb support 100. Typical of the fastening devices 120 are clamps 104, hook and loop assembly 124 and catch 130. Other fastening devices 122 may also be used.

In FIG. 6, limb support 100 is secured to second right rail 116 and second left rail 118. Here, limb support 100 serves a double function. Arm 128 is clearly supported while limb support 100 also serves as a passive restraint for the patient 106. Arm 128 in this position, may be easily treated or bandaged. Any desired bandage can be easily removed, added or changed.

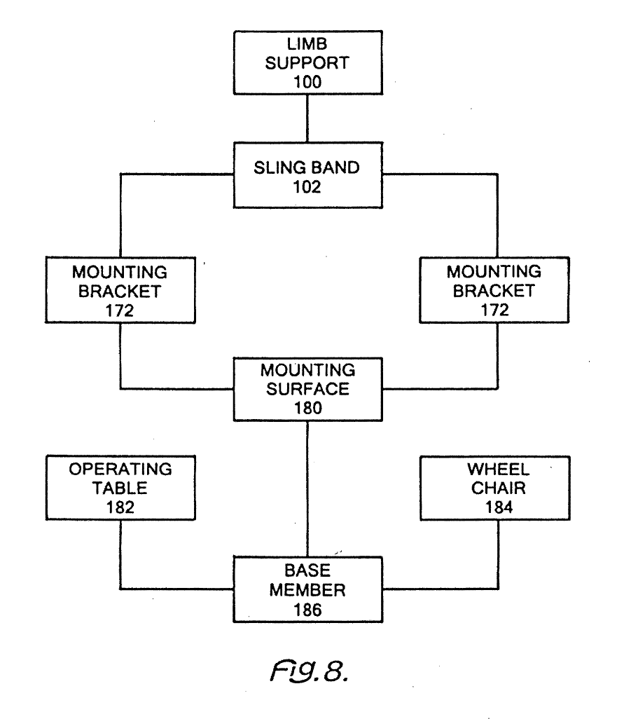

With the consideration of FIG. 7, catch 130 is secured on each end of sling band 102. With its semi flexible arcuate shaped member 132, catch 130 is easily applied to or removed from rails 112 and 114 or rails 116 and 118 by a clicking or a snapping procedure. At the same time, the care giver (not shown) may appropriately handle the limb to be treated, whether it is an arm 128 (FIG. 6) or a leg 108 (FIG. 2). The limb support 100 appears to be most efficiently usable with catch 130 secured to sling band 102.

The great utility of limb support 100 becomes even more clear with the consideration of FIG. 8. Typically, the limb support 100 includes a sling band 102 having a mounting attachment or mounting bracket 172 on either end thereof. While sling band 102 is an elongated piece of semi-rigid material, usually rectangular in shape, with this embodiment used on mounting surface 180, sling band 102 is generally wider when used on a mounting surface 180 used with a base member 186 usually available on a medical apparatus. Typically the base member 186 may a surgical operating table 182 or a wheel chair 184 or other desired surface.

When limb support 100 is used with a hospital bed 110, size of sling band 102 has certain preferred parameters. Preferably sling band 102 has a length which permits the limb support 100 to be up to about 125 percent longer than a distance between rails 112 and 114 or rails 116 and 118 is wide. More preferably sling band 102 has a length which permits the limb support 100 to be about 101 percent to about 120 percent longer than a distance between rails 112 and 114 or rails 116 and 118 is wide. Most preferably sling band 102 has a length which permits the limb support 100 to be about 102 percent to about 110 percent longer than a distance between rails 112 and 114 or rails 116 and 118 is wide.

For a width parameter sling band 102 has a width of about 10 centimeters to about 40 centimeters. More preferably sling band 102 has a width of about 11 centimeters to about 35 centimeters. Most preferably sling band 102 has a width of about 12 centimeters to about 30 centimeters.

When limb support 100 is used with a mounting surface 180, such as a wheel chair 184 or an operating table 182, for a width parameter sling band 102 has a width of about 15 centimeters to about 60 centimeters. More preferably sling band 102 has a width of about 20 centimeters to about 50 centimeters. Most preferably sling band 102 has a width of about 25 centimeters to about 40 centimeters.

This application—taken as a whole with the abstract, specification, claims, and drawings—provides sufficient information for a person having ordinary skill in the art to practice the invention disclosed and claimed herein. Any measures necessary to practice this invention are well within the skill of a person having ordinary skill in this art after that person has made a careful study of this disclosure.

Because of this disclosure and solely because of this disclosure, modification of this tool can become clear to a person having ordinary skill in this particular art. Such modifications are clearly covered by this disclosure.

What is claimed and sought to be protected by Letters Patent is:

1. A limb support to facilitate caring for a patient comprising:
a) the limb support having a sling band with a width of about 10 centimeters to about 40 centimeters to support a limb and sufficient length for mounting in a first desired area, and a width of about 15 centimeters to about 60 centimeters to support a limb and sufficient length for mounting in a second desired area;
b) a first mounting attachment at a first end of the sling band;
c) a second mounting attachment at a second end of the sling band; and
d) the first mounting attachment and the second mounting attachment cooperating to support the sling band and the limb support in the desired area.

2. The limb support of claim 1 further comprising:
a) the first desired area being a hospital bed;
b) the first mounting attachment and the second mounting attachment being attachable to a left rail and a right rail on the hospital bed;
c) the sling band receiving a limb of a patient as a supported limb; and
d) the supported limb being capable of receiving a treatment while supported on the sling band.

3. The limb support of claim 2 further comprising:
a) the first mounting attachment and the second mounting attachment including a first spring clamp and a second spring clamp;
b) the first spring clamp and the second spring clamp both receiving a first double thickness of the sling band and a second double thickness of the sling band;
c) the first double thickness of the sling band being formed by wrapping a first end of the sling band around the left rail; and
d) the second double thickness of the sling band being formed by wrapping a second end of the sling band around the right rail.

4. The limb support of claim 2 further comprising:
a) the first mounting attachment and the second mounting attachment including a first hook and loop assembly and a second hook and loop assembly;
b) the first hook and loop assembly receiving a first double thickness of the sling band;
c) the second hook and loop assembly receiving a second double thickness of the sling band;
d) the first double thickness of the sling band being formed by wrapping a first end of the sling band around the left rail; and
e) the second double thickness of the sling band being formed by wrapping a second end of the sling band around the right rail.

5. The limb support of claim 2 further comprising:
a) the first mounting attachment being a first catch cooperating with the left rail; and
b) the second mounting attachment being a second catch cooperating with the left rail.

6. The limb support of claim 5 further comprising the limb support being a passive restraint.

7. The limb support of claim 5 further comprising:
a) the first catch and the second catch each including a semi flexible arcuate shaped member;
b) the semi flexible arcuate shaped member clicking or snapping onto a the left rail or the right rail; and
c) the sling band having a sufficient length to permit the first catch to reach the left rail and the second catch to reach the right rail.

8. The limb support of claim 5 further comprising:
a) the first catch and the second catch each including a semi flexible arcuate shaped member;
b) the semi flexible arcuate shaped member clicking or snapping onto the left rail or the right rail; and
c) the sling band having a sufficient length to permit the first catch to reach the right rail and the second catch to reach the left rail.

9. A medical support device to facilitate caring for a patient comprising:
a) the limb support having a sling band with a sufficient width to support a limb and sufficient length for mounting in a desired area;
b) a first mounting attachment at a first end of the sling band;
c) a second mounting attachment at a second end of the sling band; and
d) the first mounting attachment and the second mounting attachment cooperating to support the sling band and the limb support in the desired area so that the medical support device might receive the limb as a supported limb for a treatment.

10. The medical support device of claim 9 further comprising:
a) the first mounting attachment and the second mounting attachment cooperating to support the sling band and the limb support on the desired area in form of a mounting surface used with a base member;
b) the first mounting attachment and the second mounting attachment being attachable to a left rail and a right rail on the hospital bed;
c) the sling band being adapted to receive a limb of a
d) the supported limb being capable of receiving a treatment while supported on the sling band.

11. The medical support device of claim 10 further comprising:
a) the sling band being rectangular in shape;
b) the sling band being formed of a semi rigid material;
c) the sling band having a first short side parallel to a second short side;
d) the first mounting attachment being secured to the first short side; and
e) the second mounting attachment being secured to the second short side.

12. The medical support device of claim 11 further comprising:
a) the medical support device being suitable for use on the hospital bed;
b) the hospital bed having a first set of side rails on each side thereof;
c) the first set of side rails including a first left rail oppositely disposed from a first right rail with a first distance therebetween;
d) the hospital bed having a second set of side rails on each side thereof;
e) the second set of side rails including a second left rail oppositely disposed from a second right rail with a second distance therebetween;
f) the sling band having a length of up to about 125% longer than the first distance or the second distance; and
g) the sling band having the first short side and the second short side at a width of about 10 centimeters to about 40 centimeters.

13. The medical support device of claim 12 further comprising:
a) the sling band having a length about 102 percent to about 110% longer than the first distance or the second distance; and
b) the sling band having the first short side and the second short side at a width of about 12 centimeters to about 30 centimeters, when the desired area is a hospital bed and a width of about 20 centimeters to about 50 centimeters to support a limb and sufficient length for mounting in a desired area, when the desired area is a wheel chair or operating table.

14. The medical support device of claim 9 further comprising:
a) the desired area being in the form a mounting surface used with a base member;
b) the first mounting attachment and the second mounting attachment being attachable to the mounting surface;
c) the sling band being adapted to receive a limb of a patient as a supported limb; and
d) the supported limb being capable of receiving a treatment while supported on the sling band.

15. The medical support device of claim 14 further comprising:
a) the sling band being rectangular in shape;
b) the sling band being formed of a semi rigid material;
c) the sling band having a first short side parallel to a second short side;
d) the first mounting attachment being secured to the first short side;
e) the second mounting attachment being secured to the second short side;
f) the sling band having a length of up to about 125% longer than the first distance or the second distance; and
g) the sling band having the first short side and the second short side at a width of of about 25 centimeters to about 40 centimeters to support a limb and sufficient length for mounting in a desired area, when the desired area is a wheel chair or an operating table.

16. The medical support device of claim 15 further comprising;
a) the sling band having a length about 102 percent to about 110% longer than the first distance or the second distance; and
b) the sling band having the first short side and the second short side at a width a width of about 25 centimeters to about 40 centimeters.

17. The medical support device of claim 16 further comprising the desired area being a surgical operating table or a wheel chair.

18. A method of supporting a limb for a medical procedure comprising;
a) providing a limb support with a sling band having a first mounting attachment at a first end of the sling band and a second mounting attachment at a second end of the sling band;
b) attaching the first mounting attachment and the second mounting attachment to opposing sides of a medical apparatus;
c) supporting a limb of a patient on the limb support to form a supported limb; and
d) performing a medical procedure on the supported limb.

19. The method of claim 18 further comprising:
a) the medical apparatus being a hospital bed, a wheelchair or surgical operating table; and
b) the medical procedure being an application of a bandage, a removal of a bandage, or an intravenous treatment.

20. The method of claim 19 further comprising a provision of the sling having a desired rectangular shape and semi rigid material.
